(19) United States
(12) Patent Application Publication
(10) Pub. No.: US 2017/0000558 A1
(43) Pub. Date: Jan. 5, 2017

(54) MEDICAL TREATMENT APPARATUS

(71) Applicant: OLYMPUS CORPORATION, Tokyo (JP)

(72) Inventor: Hikaru INOUE, Higashimurayama-shi (JP)

(73) Assignee: OLYMPUS CORPORATION, Tokyo (JP)

(21) Appl. No.: 15/268,557

(22) Filed: Sep. 17, 2016

Related U.S. Application Data
(63) Continuation of application No. PCT/JP2015/056706, filed on Mar. 6, 2015.

(30) Foreign Application Priority Data
Apr. 18, 2014 (JP) 2014-086538

(51) Int. Cl. A61B 18/14; A61N 7/02; A61B 17/32

(52) U.S. Cl. CPC A61B 18/1445; A61B 17/320092; A61N 7/02; A61B 2018/00589

(57) ABSTRACT

A medical treatment apparatus includes a first holding member, and a second holding member configured to move relative to the first holding member and hold a living tissue between the first holding member and the second holding member. On a cross section including a point where the first and second holding member are in contact with the living tissue, a point on the first holding member that is nearest to the second holding member is defined as a first point and a point on the second holding member that is nearest to the first holding member is defined as a second point. The first and second holding member have shapes in which an extended line of a trace of the second point moving relative to the first holding member does not pass through the first point.

### CROSS-REFERENCE TO RELATED APPLICATIONS

This application is a Continuation Application of PCT Application No. PCT/JP2015/056706, filed Mar. 6, 2015 and based upon and claiming the benefit of priority from prior Japanese Patent Application No. 2014-086538, filed Apr. 18, 2014, the entire contents of all of which are incorporated herein by reference.

### BACKGROUND OF THE INVENTION

The present invention relates to a medical treatment apparatus.

### DESCRIPTION OF THE RELATED ART

A mechanical treatment tool, such as a stapler and an energy-application treatment tool, are known as medical treatment tools to join living tissues. When a living tissue is joined to another living tissue by an energy-application treatment tool, the living tissues are held by a holding portion comprising a pair of jaws and joined by energy applied to the holding portion.

For example, Jpn. Pat. Appln. KOKAI Publication No. 2012-239899 discloses a technique relating to a surgical treatment tool that seals a blood vessel and a vessel channel tissue by applying electrical energy and physical energy to the blood vessel and the vessel channel tissue and facilitating extraction of collagen and elastin.

Generally, when a living tissue is joined to another living tissue by an energy-application treatment tool, it is necessary to apply energy to the living tissues to be joined that are in close contact with each other to obtain a sufficient joint strength. Particularly, in organs that contain a large amount of digestive fluid or mucous membrane, such as digestive system organs, a sufficient joint strength may not be obtained because of the presence of undigested material, digestive fluid, or mucous membrane between the living tissues.

### BRIEF SUMMARY OF THE INVENTION

According to an aspect of the invention, a medical treatment apparatus includes a first holding member; and a second holding member configured to move relative to the first holding member and hold a living tissue between the first holding member and the second holding member, wherein, on a cross section including a point where the first holding member and the second holding member are in contact with the living tissue, where a point on the first holding member that is nearest to the second holding member is defined as a first point and a point on the second holding member that is nearest to the first holding member is defined as a second point, the first holding member and the second holding member have shapes in which an extended line of a trace of the second point moving relative to the first holding member does not pass through the first point.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic diagram showing a configuration example of treatment apparatuses according to embodiments of the present invention;

FIG. 2A is a schematic perspective diagram of a treatment portion according to a first embodiment;

FIG. 2B is a schematic diagram showing a state of the treatment portion which is holding living tissues, as viewed from a distal end side of the treatment portion, according to the first embodiment;

FIG. 3A is a schematic perspective diagram of a treatment portion of a comparative example;

FIG. 3B is a schematic diagram showing a state of the treatment portion which is holding living tissues, as viewed from a distal end side of the treatment portion, according to the comparative example;

FIG. 4A is a diagram for explaining a shape of the treatment portion according to the first embodiment;

FIG. 4B is a diagram for explaining a shape of the treatment portion according to the first embodiment;

FIG. 5A is a schematic perspective diagram of a treatment portion according to a first modification of the first embodiment;

FIG. 5B is a schematic diagram showing a state of the treatment portion which is holding living tissues, as viewed from a distal end side of the treatment portion, according to the first modification of the first embodiment;

FIG. 6A is a schematic perspective diagram of a treatment portion according to a second modification of the first embodiment;

FIG. 6B is a schematic diagram showing a state of the treatment portion which is holding living tissues, as viewed from a distal end side of the treatment portion, according to the second modification of the first embodiment;

FIG. 7A is a schematic diagram showing a state of a treatment portion which is holding living tissues, as viewed from a distal end side of the treatment portion, according to a third modification of the first embodiment;

FIG. 7B is a schematic diagram showing the treatment portion according to the third modification of the first embodiment, as viewed from a distal end side of the treatment portion;

FIG. 8 is a diagram for explaining angles of inclinations provided in the treatment portion according to the third modification of the first embodiment;

FIG. 9 is a schematic diagram showing a treatment portion according to a fourth modification of the first embodiment, as viewed from a distal end side of the treatment portion;

FIG. 10A is a schematic diagram showing a treatment portion according to a fifth modification of the first embodiment, as viewed from a distal end side of the treatment portion;

FIG. 10B is a schematic diagram showing a treatment portion according to a sixth modification of the first embodiment, as viewed from a distal end side of the treatment portion;

FIG. 11A is a schematic perspective diagram of a treatment portion according to a second embodiment;

FIG. 11B is a diagram for explaining a cross-sectional shape of the treatment portion according to the second embodiment;

FIG. 11C is a diagram for explaining a cross-sectional shape of the treatment portion according to the second embodiment;

FIG. 12A is a schematic perspective diagram of a treatment portion according to a third embodiment; and

FIG. 12B is a diagram for explaining a cross-sectional shape of the treatment portion according to the third embodiment.

### DETAILED DESCRIPTION OF THE INVENTION

### First Embodiment

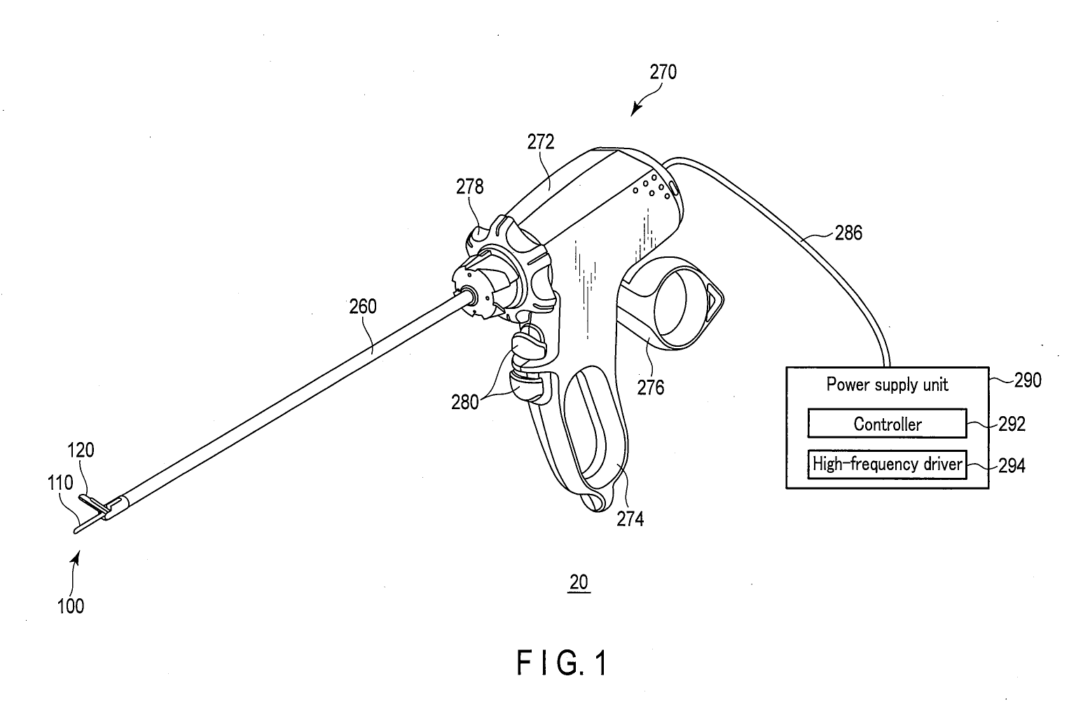

The first embodiment of the present invention will be explained with reference to the drawings. FIG. 1 is a schematic diagram showing a medical treatment apparatus 20 according to the embodiment. As shown in the figure, the treatment apparatus 20 comprises a treatment portion 100, a shaft 260, an operating portion 270, and a power supply unit 290. In the following, for the purpose of explanation, a side of the treatment portion 100 is referred to as a distal end side, and a side of the operating portion 270 is referred to as a proximal end side. The treatment apparatus 20 holds living tissues as a treatment target with the treatment portion 100, and applies a high-frequency voltage to the held living tissues, thereby sealing or coagulating the living tissues.

The treatment portion 100 is provided at a distal end of the shaft 260. The treatment portion 100 comprises a pair of jaws that change positions relative to each other to hold living tissues. One of the jaws is referred to as a first holding member 110 and the other is referred to as a second holding member 120. In this embodiment, a part of the first holding member 110 and a part of the second holding member 120 function as bipolar electrodes that apply a high-frequency voltage to a held living tissue. However, the embodiment is not limited to this configuration but may be configured so that a part of the first holding member 110 or a part of the second holding member 120 functions as a monopolar electrode.

The operating portion 270 comprises an operating portion main body 272, a fixed handle 274, a movable handle 276, a rotating knob 278 and an output switch 280. The fixed handle 274 is fixed to the operating portion main body 272, whereas the movable handle 276 changes its position relative to the operating portion main body 272. The movable handle 276 is connected to a wire or a rod, which is connected to the first holding member 110 and the second holding member 120 through the shaft 260. Operations of the movable handle 276 are transmitted to the first holding member 110 and the second holding member 120 via the wire or the rod. The first holding member 110 and the second holding member 120 change their relative positions in accordance with the operations of the movable handle 276. The rotating knob 278 is a knob to rotate a part extending from the rotating knob 278 to the distal end side. The shaft 260 rotates in accordance with rotation of the rotating knob 278 to adjust an angle of the treatment portion 100.

The output switch 280 includes, for example, a button switch. When the button switch is pushed, a high-frequency voltage is applied to the living tissues held by the treatment portion 100. As a result, the living tissues held by the treatment portion 100 are joined to each other.

One end of a cable 286 is connected to the proximal end side of the operating portion 270. The other end of the cable 286 is connected to the power supply unit 290. The power supply unit 290 includes a controller 292 and a high-frequency driver 294. The controller 292 controls various parts of the treatment apparatus 20. For example, the controller 292 controls an operation of the high-frequency driver 294 in accordance with an input from the output switch 280. The high-frequency driver 294 supplies a high-frequency current to the treatment portion 100 under the control of the controller 292.

An operation of the treatment apparatus 20 according to the embodiment is explained below. An operator sets output conditions of the treatment apparatus, for example, setting an output power of high-frequency energy, treatment time, etc., by operating the input portion of the power supply unit 290. The treatment apparatus 20 may be configured so that values of the conditions can be individually set, or a set of setting values according to an operative method can be selected.

The treatment portion 100 and the shaft 260 are inserted, for example, through an abdominal wall into an abdominal cavity. The operator operates the movable handle 276 to open and close the treatment portion 100, and holds living tissues as a treatment target with the first holding member 110 and the second holding member 120. The operator operates the output switch 280 upon holding the living tissues with the treatment portion 100. When the button is pushed, the controller 292 of the power supply unit 290 outputs driving-related instructions to the high-frequency driver 294.

The high-frequency driver 294 applies a high-frequency voltage to the first holding member 110 and to the second holding member 120 of the treatment portion 100 under the control of the controller 292, to cause the high-frequency current to flow to the living tissues as the treatment target. Since the living tissues serve as an electric resistor when the high-frequency current flows therethrough, heat is generated in the living tissues and the temperature of the living tissues rises. As a result, proteins in the living tissues denature, and accordingly the living tissues are coagulated and sealed. Thus, the operation of joining the living tissues is completed.

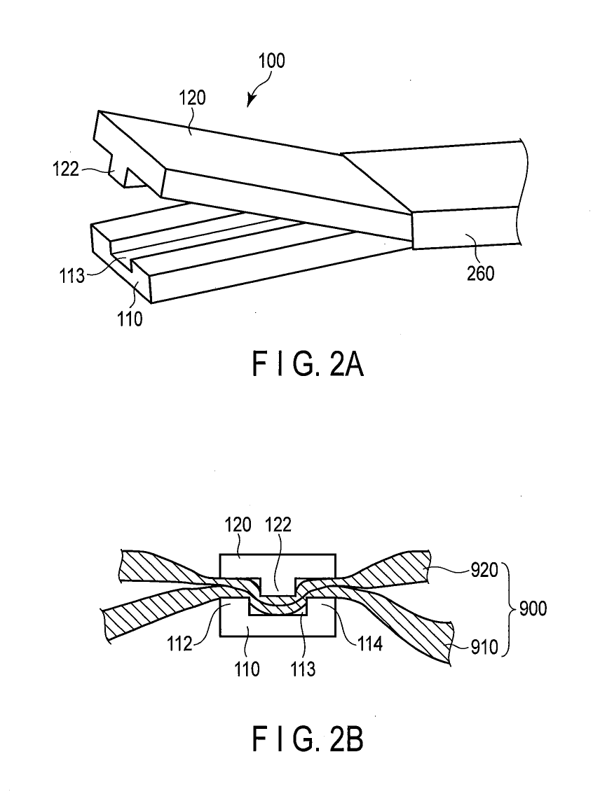

A structure of the treatment portion 100 will be detailed below. FIG. 2A is a schematic perspective diagram showing a structure of the treatment portion 100 according to this embodiment. FIG. 2B is a schematic diagram showing a state of the treatment portion 100 holding living tissues 900 as the treatment target, as viewed from the distal end side of the treatment portion 100. As shown in FIG. 2A and FIG. 2B, the first holding member 110 is provided with a concave part 113 in a surface facing the second holding member 120. On the other hand, the second holding member 120 is provided with a convex part 122 in a surface facing the first holding member 110. The convex part 122 of the second holding member 120 is configured to fit inside the concave part 113 of the first holding member 110.

The treatment portion 100 of this embodiment clips two pieces of living tissues to be joined together. As shown in FIG. 2B, of the living tissues 900 held by the treatment portion 100, one that is located on a side of the first holding member 110 is referred to as a first tissue 910, whereas one that is located on a side of the second holding member 120 is referred to as a second tissue 920. When the living tissues 900 are held by the treatment portion 100, the first tissue 910 and the second tissue 920 are pressed against each other.

As shown in FIG. 2B, a central portion of the second holding member 120 including the convex part 122 presses down a central portion of the living tissues 900 toward a bottom of the drawing. On the other hand, a right convex part 112 and a left convex part 114 of the first holding member 110 that are located right and left of the concave part 113 press right and left portions of the living tissues 900 upward, as shown in FIG. 2B. As a result, when the living tissues 900 are gradually compressed, shearing stress occurs in joint surfaces, which are interfaces of the first tissue 910 and the second tissue 920. Accordingly, in a part where the shearing stress occurs, materials sandwiched between the first tissue 910 and the second tissue 920 are pushed out to outside of the treatment portion 100. The materials sandwiched between the first tissue 910 and the second tissue 920 are, for example, undigested material, digestive fluid, or mucous membrane on an internal surface of an organ. In the explanation herein, both the first holding member 110 and the second holding member 120 move. However, since it is acceptable merely that the living tissues 900 be held by a change of the relative positions of the first holding member 110 and the second holding member 120, moving at least one of the first holding member 110 and the second holding member 120 is acceptable.

After the living tissues 900 are compressed while the materials sandwiched between the first tissue 910 and the second tissue 920 are pressed out, a high-frequency voltage is applied to the held living tissues 900. As a result, the first tissue 910 and the second tissue 920 are joined together at the interfaces thereof.

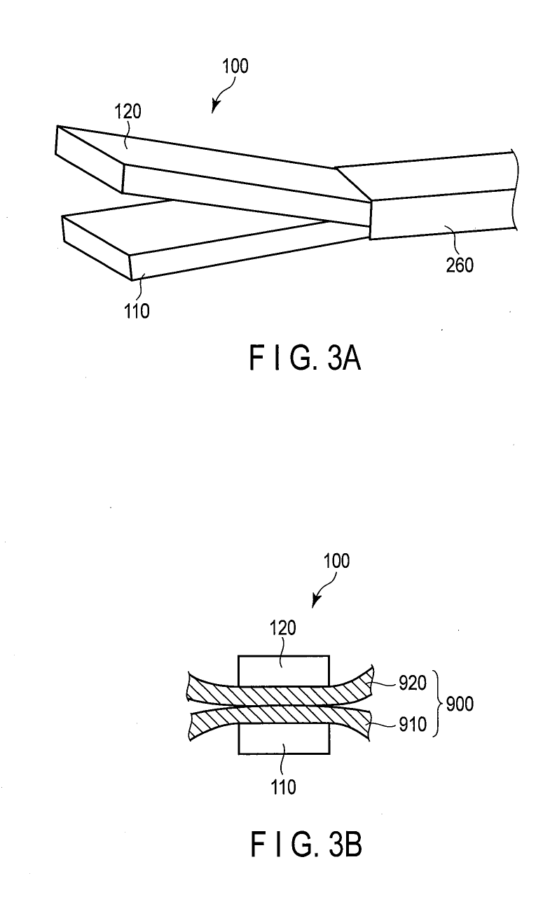

To achieve a tight energy joint, it is necessary to stimulate three dimensional entanglement of extracellular matrices of an organ. Therefore, facing tissues need to be brought into close contact with each other directly. A comparative example shown in FIG. 3A is considered, for example, in which facing surfaces of the first holding member 110 and the second holding member 120 are flat surfaces. In the comparative example, when the treatment portion 100 holds the first tissue 910 and the second tissue 920 laid one on another, the first tissue 910 and the second tissue 920 form shapes as shown in FIG. 3B. As a result, it is likely that the materials present between the first tissue 910 and the second tissue 920 are not pushed out, but are easily sandwiched between the first tissue 910 and the second tissue 920. In this case, the first tissue and the second tissue cannot be directly brought into close contact with each other. Accordingly, the joint strength may be low. In contrast, according to the treatment portion 100 of the embodiment, since the materials sandwiched between the first tissue 910 and the second tissue 920 are pressed out, the first tissue 910 and the second tissue 920 are brought into close contact at the interfaces thereof and can achieve a joint with sufficient strength.

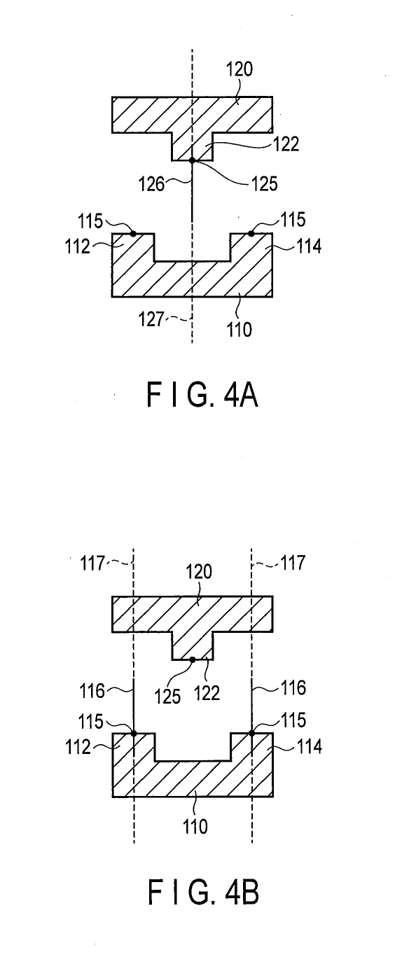

Cross-sectional shapes of the first holding member 110 and the second holding member 120 will be described with reference to FIG. 4A and FIG. 4B. FIG. 4A and FIG. 4B show shapes of the first holding member 110 and the second holding member 120 in a cross section where the first holding member 110 and the second holding member 120 are in contact with living tissues. The cross sections shown in FIG. 4A and FIG. 4B are planes perpendicular to a longitudinal axis of the first holding member 110 and the second holding member 120, as viewed from the distal end side. As shown in FIG. 4A, a point (surface) on the first holding member 110 that is nearest to the second holding member 120 is defined as a first point 115. In the embodiment, those surfaces of the right convex part 112 and the left convex part 114 of the first holding member 110 that face the second holding member 120 have planar shapes, which can be at a constant distance from the second holding member 120. Accordingly, there are an infinite number of first points on the right convex part 112 and the left convex part 114. However, in the drawings, one of first points 115 on each of the right convex part 112 and the left convex part 114 is shown as a representative. Furthermore, a point on the second holding member 120 that is nearest to the first holding member 110 is defined as a second point 125. Similarly, one of the second points 125 is shown as a representative.

When the second holding member 120 moves relative to the first holding member 110, the second point 125 traces a solid line 126 shown in FIG. 4A. As indicated by a broken line 127, the extended line of the solid line 126 does not pass through the first points 115.

The same applies to the first points 115, since the first holding member 110 and the second holding member 120 move relative to each other. Specifically, as shown in FIG. 4B, the first points 115 trace solid lines 116 shown in FIG. 4B. As indicated by broken lines 117, the extended lines of the solid lines 116 do not pass through the second point 125. In other words, when the first holding member 110 and the second holding member 120 hold living tissues, the first points 115 (and the surfaces including the same) and the second point 125 (and the surface including the same) are kept in non-contact with each other. In more detail, the traces of the first points 115 and the second point 125 cross a normal line perpendicular to the traces during relative movement, but keep a non-contact state with respect to a line parallel to the traces. In other words, during relative movement, the traces of the first points 115 may cross a normal line perpendicular to the trace of the second point 125, but cannot cross the trace of the second point 125. Furthermore, during relative movement, the trace of the second point 125 crosses a normal line perpendicular to the traces of the first points 115, but does not cross the traces of the first points 115.

Because of the shapes of the first holding member 110 and the second holding member 120 as described above, shearing stress occurs in joint surfaces of the living tissues 900 held by the first holding member 110 and the second holding member 120.

Treatment targets of the treatment apparatus 20 of the embodiment are various tissues such as a small intestine, a large intestine, a stomach, a blood vessel, and a lymphatic vessel. An advantage of the embodiment of improving the joint strength is obtainable especially in an organ that contains a large amount of undigested material, digestive fluid, or mucous membrane, such as an organ in the digestive system. Since vessels, for example, blood vessels and lymphatic vessels, discharge fluid such as blood or lymphatic fluid, the advantage of the embodiment of improving the joint strength is also obtainable in the vessels.

A depth of the concave part 113 of the first holding member 110 and a height of the convex part 122 of the second holding member 120 will now be described. For example, it is assumed that treatment targets are tissues in a stomach. Mucous membrane of the stomach is about 1 mm and a tissue thickness of the stomach is about 5 mm. When two tissues are joined, the thickness in total is about 10 mm. The treatment portion 100 holds the approximately 10 mm thick tissues. The tissue thickness widely varies depending on the organ. The tissue of the stomach is comparatively thick. In contrast, the tissue of a small intestine is relatively thin, that is, about 2 mm. In the case of the small intestine, the thickness of two tissues in total is about 4 mm.

When the two tissues held by the treatment portion 100 are compressed, it is preferable to compress the tissues about 20% of the thickness. Therefore, it is preferable that the depth of the concave part 113 of the first holding member 110 and the height of the convex part 122 of the second holding member 120 be about 20% of the thickness of the two pieces of the living tissues to be treated.

Also, mucous membrane has a complicated structure. It is assumed that contents having entered the complicated structure are pressed out. In this case, it is preferable that the depth of the concave part 113 of the first holding member 110 and the height of the convex part 122 of the second holding member 120 be approximately equal to the thickness of the two pieces of the mucous membrane. For example, in the case of treating the small intestine, since the thickness of the mucous membrane in the small intestine is about 200-300 μm, it is preferable that the depth of the concave part 113 of the first holding member 110 and the height of the convex part 122 of the second holding member 120 be about 400-600 μm.

The thickness of a tissue and the thickness of the mucous membrane vary from tissue to tissue; therefore, the shape and size of the treatment portion 100 may be appropriately determined in accordance with the treatment target. The amount of a gap between the first point 115 and the second point 125 may also be appropriately adjusted in accordance with the thickness of tissues. For example, a sufficient shear stress can be generated by setting the amount of a gap to the thickness of the tissues to be treated or greater.

In this embodiment, the first holding member 110 has a concave shape and the second holding member 120 has a convex shape. However, the concavity and the convexity may be reversed. That is, the first holding member 110 may have a convex shape and the second holding member 120 may have a concave shape. In other words, either the first holding member 110 or the second holding member 120 may be located in the upper or lower side of the treatment portion 100.

The embodiment has been explained with respect to an apparatus that joins living tissues by applying high-frequency power to the living tissues. However, the energy applied to the living tissues may be of any kind of energy. For example, thermal energy may be applied to living tissues by heating the living tissues with heaters provided in the first holding member 110 and the second holding member 120. Also, for example, the first holding member 110 may comprise an ultrasonic probe to heat living tissues by ultrasonic vibrations. Besides, the treatment portion 100 may be configured to treat living tissues held in various methods. The configuration of the treatment portion 100 and the configuration of the power supply unit 290 may be appropriately changed in accordance with the kind of energy applied to the living tissues.

[First Modification of First Embodiment]

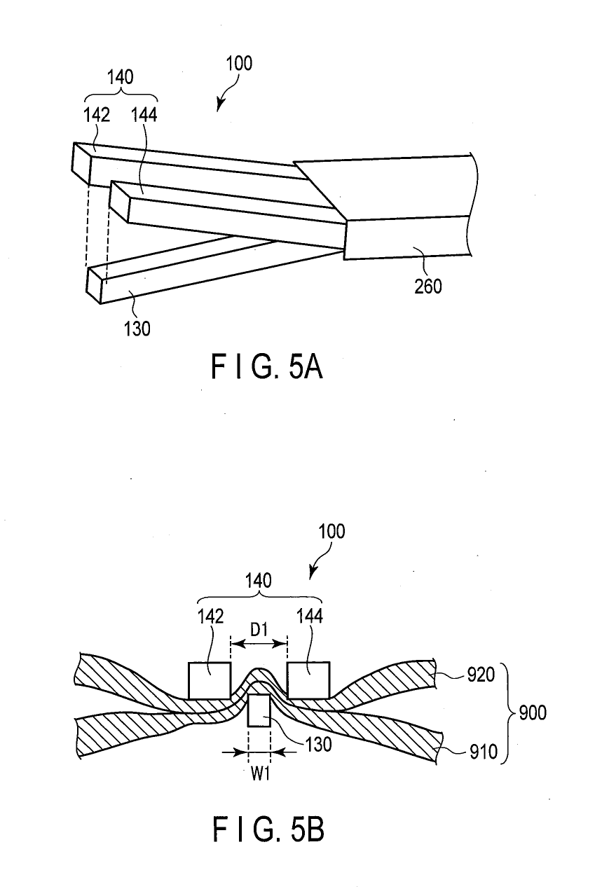

The first modification of the first embodiment is explained below. In the following, matters different from the first embodiment will be explained. The same symbols as used in the first embodiment will be used for the same parts, and detailed explanations thereof will be omitted. In this modification, a pair of holding members of the treatment portion 100 have a shape different from that of the first embodiment. A shape of the treatment portion 100 of this modification will be described with reference to FIG. 5A and FIG. 5B.

FIG. 5A is a schematic perspective diagram showing a structure of the treatment portion 100 according to this modification. FIG. 5B is a schematic diagram showing a state of the treatment portion 100 holding living tissues 900 to be treated, as viewed from the distal end side of the treatment portion 100. As shown in the figures, the treatment portion 100 of the modification comprises a third holding member 130 and a fourth holding member 140. The third holding member 130 and the fourth holding member 140 face each other. Living tissues 900 to be treated are held between the third holding member 130 and the fourth holding member 140. Thus, the third holding member 130 and the fourth holding member 140 move relative to each other, and function as the first holding member and the second holding member, which hold living tissues therebetween.

The fourth holding member 140 comprises a right member 142 and a left member 144. A predetermined distance D1 is provided between the right member 142 and the left member 144. The right member 142 and the left member 144 maintain a positional relationship therebetween, while moving toward the third holding member 130.

A width W1 of the third holding member 130 is smaller than the distance D1 between the right member 142 and the left member 144. When the third holding member 130 moves toward the fourth holding member 140, the third holding member 130 will be located between the right member 142 and the left member 144.

In this modification, as well as the first embodiment, shearing stress occurs in joint surfaces, which are interfaces of a first tissue 910 and a second tissue 920 of living tissues 900 held by the treatment portion 100. Accordingly, in a part where the shearing stress occurs, materials sandwiched between the first tissue 910 and the second tissue 920 are pressed out. As a result, the first tissue 910 and the second tissue 920 can be joined at the interfaces with sufficient strength.

Either the third holding member 130 or the fourth holding member 140 may be located in either the upper or lower side of the treatment portion 100. FIG. 5B shows an example, in which the third holding member 130 is placed in a position corresponding to the center of the fourth holding member 140. The modification is not limited to this example; that is, the third holding member 130 may be nearer to either the right member 142 or the left member 144. Including such a case, the treatment portion 100 may be asymmetric.

[Second Modification of First Embodiment]

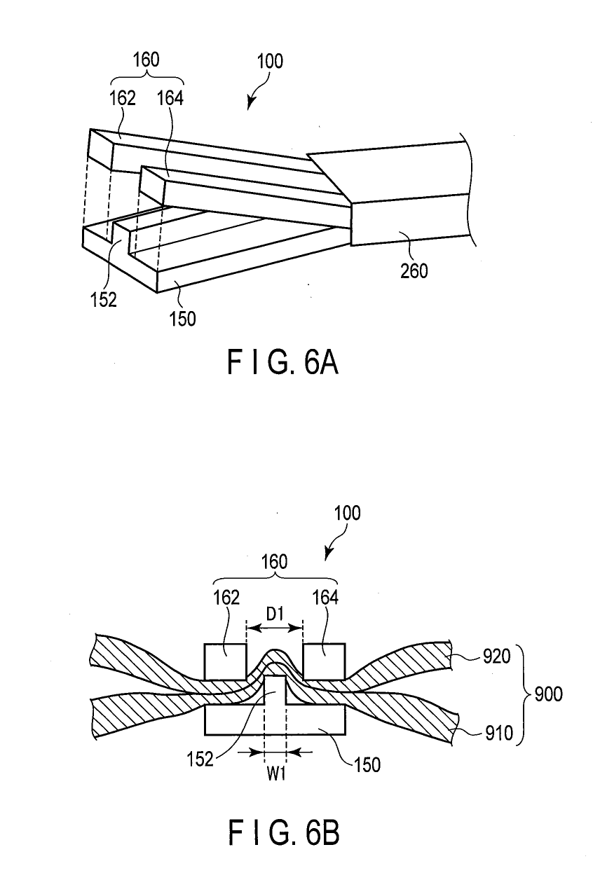

The second modification of the first embodiment is explained below. In the following, matters different from the first embodiment will be explained. Identical symbols will be used for identical parts, and detailed explanations thereof will be omitted. In this modification, a pair of holding members of the treatment portion 100 has a shape different from that of the first embodiment. A shape of the treatment portion 100 of this modification will be described with reference to FIG. 6A and FIG. 6B.

FIG. 6A is a schematic perspective diagram showing a structure of the treatment portion 100 according to this embodiment. FIG. 6B is a schematic diagram showing a state of the treatment portion 100 holding living tissues 900 to be treated, as viewed from the distal end side of the treatment portion 100. As shown in the figures, the treatment portion 100 of the modification comprises a fifth holding member 150 and a sixth holding member 160. The fifth holding member 150 and the sixth holding member 160 face each other. Living tissues 900 to be treated are held between the fifth holding member 150 and the sixth holding member 160. Thus, the fifth holding member 150 and the sixth holding member 160 move relative to each other, and function as the first holding member and the second holding member, which hold living tissues therebetween.

The sixth holding member 160, as well as the first modification, comprises a right member 162 and a left member 164. A predetermined distance D1 is provided between the right member 162 and the left member 164. The right member 162 and the left member 164 maintain the positional relationship therebetween, while moving toward the fifth holding member 150.

The fifth holding member 150 has a convex part 152. A width W1 of the convex part 152 is smaller than the distance D1 between the right member 162 and the left member 164. When the fifth holding member 150 moves toward the sixth holding member 160, the convex part 152 of the fifth holding member 150 will be located between the right member 162 and the left member 164.

In this modification, as well as the first embodiment, shearing stress occurs in joint surfaces, which are interfaces of a first tissue 910 and a second tissue 920 of living tissues 900 held by the treatment portion 100. Accordingly, in a part where the shearing stress occurs, materials sandwiched between the first tissue 910 and the second tissue 920 are pressed out. As a result, the first tissue 910 and the second tissue 920 can be joined at the interface with sufficient strength.

Either the fifth holding member 150 or the sixth holding member 160 may be located in the upper or lower side of the treatment portion 100. Furthermore, the treatment portion 100 may be asymmetric in a right and left direction.

[Third Modification of First Embodiment]

The third modification of the first embodiment is explained below. In the following, matters different from the first modification of the first embodiment will be explained. Identical symbols will be used for identical parts, and detailed explanations thereof will be omitted. In this modification, those parts of the holding members of the treatment portion 100 that face each other have inclinations.

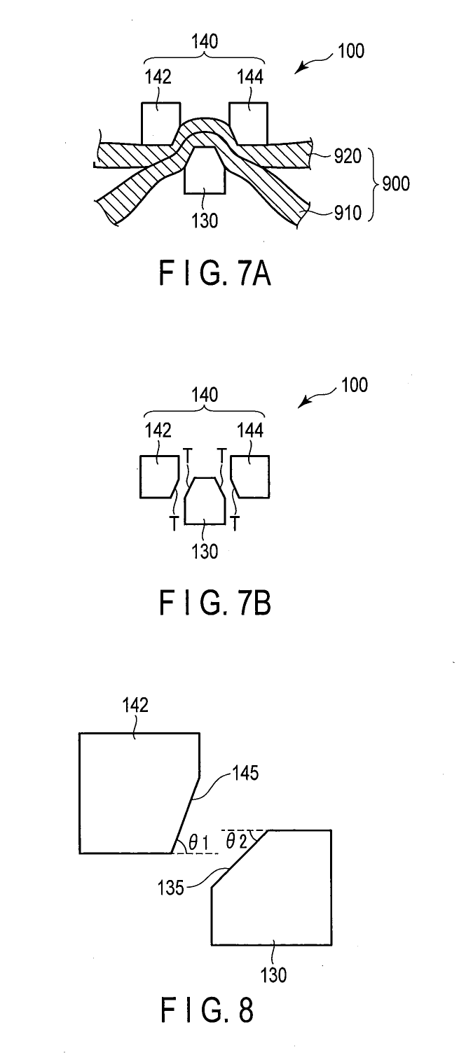

FIG. 7A is a schematic diagram showing a state of the treatment portion 100 of the modification holding living tissues 900 to be treated, as viewed from the distal end side of the treatment portion 100. FIG. 7B is a schematic diagram showing a state of the treatment portion 100 as viewed from the distal end side of the treatment portion, where there are no living tissues 900. As shown in the figures, the treatment portion 100 of the modification comprises a third holding member 130, and a fourth holding member 140 which includes a right member 142 and a left member 144. Living tissues 900 are held between the third holding member 130 and the fourth holding member 140.

Each of corners of the third holding member 130 and the right member 142 and the left member 144 of the fourth holding member 140, which are brought into contact with living tissues, has an inclination T. Because of such a shape, shearing stress that occurs at the held living tissues is more stable as compared to a case in which there is no inclination.

The inclinations T may have an angle that makes the surfaces facing each other parallel. Even if the facing surfaces are parallel, shearing stress will occur in joint surfaces of the held living tissues 900. Alternatively, angles of the inclinations may be determined as described below. As shown in FIG. 8, the right member 142 of the fourth holding member 140 and the third holding member 130 have horizontal surfaces, which are opposing each other and not inclined. In this case, an angle θ1 of an inclination surface 145 of the right member 142 with respect to the horizontal surface is greater than an angle θ2 of an inclination surface 135 of the third holding member 130 with respect to the horizontal surface. By setting the angle θ1 and the angle θ2 as shown in FIG. 8, force acts so that the right member 142 rubs against the inclination surface 135 of the third holding member 130 while moving. As a result, materials between two joint surfaces can be pressed out more efficiently.

In this modification, inclinations are provided in the holding members according to the first modification. However, inclinations may be provided in the holding members of the first embodiment or the second modification thereof. Furthermore, as described above, the angle of an inclination is determined in accordance with the direction in which the materials between two joint surfaces of living tissues are to be pressed out.

[Fourth Modification of First Embodiment]

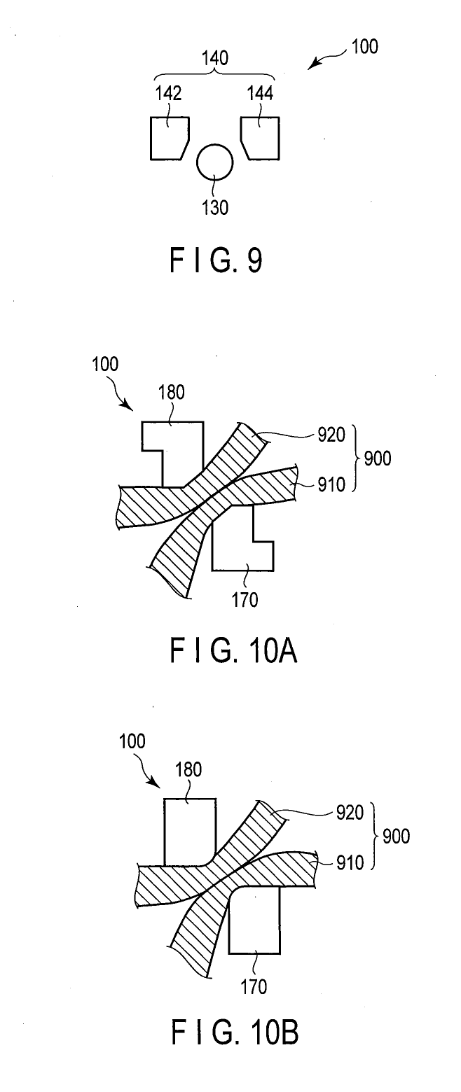

The fourth modification of the first embodiment is explained below. In the following, matters different from the first modification of the first embodiment will be explained. Identical symbols will be used for identical parts, and detailed explanations thereof will be omitted. FIG. 9 is a schematic diagram showing a treatment portion 100 according to this modification, as viewed from a distal end side of the treatment portion. As shown in the figure, a third holding member 130 of this modification has a circular cross-sectional shape. Even in this modification, in which the third holding member 130 has a circular cross-sectional shape, materials between two joint surfaces of living tissues can be pressed out and high joint strength can be realized.

In a case where the third holding member 130 comprises an ultrasonic probe, it is preferable that the cross-sectional shape thereof be, for example, nearly circular, in consideration of vibration characteristics of the third holding member. The circular cross-sectional shape of the third holding member 130 of this modification enables the third holding member 130 to be an ultrasonic probe having improved energy efficiency.

[Fifth Modification of First Embodiment]

The fifth modification of the first embodiment is explained below. In the following, matters different from the third modification of the first embodiment will be explained. Identical symbols will be used for identical parts, and detailed explanations thereof will be omitted. In the third modification of the first embodiment, shearing stress is applied to living tissues 900 in two places of the sides of the third holding member 130. In contrast, according to this modification, shearing stress is applied to living tissues 900 in one place.

FIG. 10A is a schematic diagram showing a treatment portion 100, as viewed from a distal end side. As shown in the figure, the treatment portion 100 of the modification comprises a seventh holding member 170 and an eighth holding member 180. The seventh holding member 170 and the eighth holding member 180 face each other. The living tissues 900 to be treated are held between the seventh holding member 170 and the eighth holding member 180. In this manner, the seventh holding member 170 and the eighth holding member 180 move relative to each other, and function as the first holding member and the second holding member, which hold living tissues therebetween.

A line extending in a direction of movement of the seventh holding member 170 from a position where the seventh holding member 170 acts on the living tissues 900 does not coincide with a line extending in a direction of movement of the eighth holding member 180 from a position where the eighth holding member 180 acts on the living tissues 900. In this case, shearing stress occurs in interfaces of a first tissue 910 and a second tissue 920 of the living tissues 900 held by the seventh holding member 170 and the eighth holding member 180. Accordingly, in a part where the shearing stress occurs, materials sandwiched between the first tissue 910 and the second tissue 920 are pressed out. This produces an advantage of improving the joint strength of the interfaces of the first tissue 910 and the second tissue 920.

The above matter can be rephrased as follows: A point on the seventh holding member 170 that is nearest to the eighth holding member 180 is defined as a first point, and a point on the eighth holding member 180 that is nearest to the seventh holding member 170 is defined as a second point. When the eighth holding member 180 moves relative to the seventh holding member 170, an extended line of the trace of the second point does not pass through the first point. In such a case, the advantage of the present invention is obtained.

Furthermore, in this modification, the surfaces of the seventh holding member 170 and the eighth holding member 180, which face each other, have inclinations. The seventh holding member 170 and the eighth holding member 180 are described to be symmetric, as an example. However, these members may be asymmetric.

[Sixth Modification of First Embodiment]

The sixth modification of the first embodiment is explained below. In the following, matters different from the fifth modification of the first embodiment will be explained. Identical symbols will be used for identical parts, and detailed explanations thereof will be omitted. FIG. 10B is a schematic diagram showing a treatment portion 100, as viewed from a distal end side. As shown in the figure, the opposing surfaces of a seventh holding member 170 and an eighth holding member 180 are rounded. The other configurations are the same as those of the fifth modification of the first embodiment. This modification produces the same advantage as that of the fifth modification of the first embodiment.

The treatment portion 100 configured to press out contents right and left in a cross section perpendicular to the longitudinal direction of the treatment portion 100 was described as an example of the first embodiment and the modifications thereof. However, the embodiment is not limited to the above example, but may be configured to press out contents to the right and left in a cross section parallel to the longitudinal direction of the treatment portion 100. In this case, the cross section parallel to the longitudinal direction of the treatment portion 100 may have a shape that can generate shearing stress on joint surfaces of living tissues 900 as described above. For example, a first holding member located in a lower side of the treatment portion 100 may be convex in a distal end side and concave in a proximal end side with respect to a second holding member arranged in an upper side, and the second holding member may be concave in the distal end side and convex in the proximal end side with respect to the first holding member.

As described above, in all of the first embodiment and the modifications thereof, the pair of holding members of the treatment portion 100 that face each other have a shape as described below in a cross section including a point where the holding members are in contact with living tissues. A point on one holding member that is nearest to the other holding member is defined as a first point, and a point on the other holding member that is nearest to the one holding member is defined as a second point. When the holding member moves, the trace of the second point and an extended line thereof do not pass through the first point. With this shape, in two living tissues held between the paired holding members, shearing stress occurs in surfaces of the tissues that are in contact with each other. As a result, unwanted materials existing on the surfaces of the tissues in contact with each other are pressed out and the surfaces are brought into close contact with each other. The tissues can be firmly joined together by applying energy to the tissues that are in close contact.

### Second Embodiment

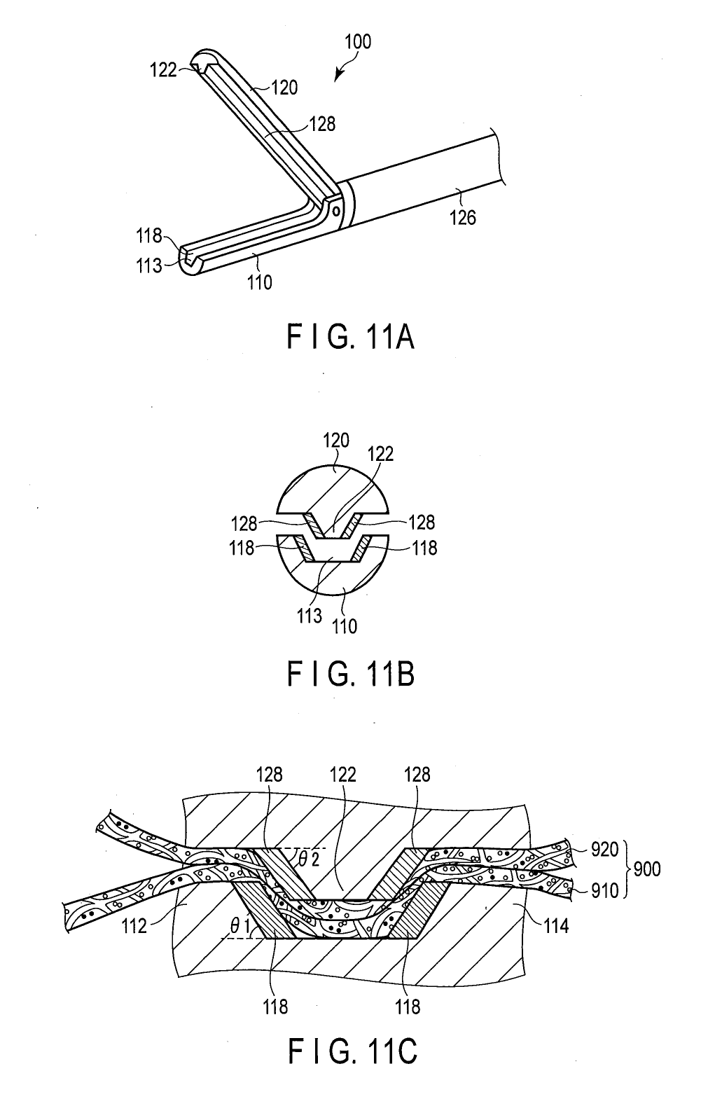

The second embodiment is explained below. In the following, matters different from the first embodiment will be explained. Identical symbols will be used for identical parts, and detailed explanations thereof will be omitted. FIG. 11A is a perspective diagram showing a treatment portion 100 of this embodiment. FIG. 11B is a cross-sectional view on a cross section perpendicular to the longitudinal direction of the treatment portion 100, as viewed from a distal end side of the treatment portion 100. Placement of an energy application portion is specifically described below.

As shown in FIG. 11A and FIG. 11B, a first holding member 110 is provided with a concave part 113, as well as the first embodiment. A second holding member 120 is provided with a convex part 122. In this embodiment, as well as the third modification of the first embodiment, surfaces of the first holding member 110 and the second holding member 120, which face each other, have inclinations. A first energy application portion 118 is provided on a surface of the inclination of the first holding member 110 that faces a surface of the inclination of the second holding member 120. A second energy application portion 128 is provided on a surface of the inclination of the second holding member 120 that faces a surface of the inclination of the first holding member 110.

The first energy application portion 118 and the second energy application portion 128 are, for example, electrodes. During treatment, high-frequency treatment is performed by application of a high-frequency voltage across the first energy application portion 118 and the second energy application portion 128 with living tissues 900 interposed therebetween. A current corresponding to the applied voltage flows through the interposed living tissues. At this time, heat is generated in the living tissues by electric resistance in the living tissues. The living tissues are joined to each other by the heat.

The first energy application portion 118 and the second energy application portion 128 may be, for example, heaters. In this case, the heaters are heated to a high temperature in the treatment, and the living tissues held by the treatment portion 100 are joined to each other by the heat.

FIG. 11C is an enlarged cross-sectional view showing an area near the concave part 113 of the first holding member 110 and the convex part 122 of the second holding member 120. As shown in the figure, angles of the inclinations provided on the first holding member 110 and the second holding member 120 are the same as those explained above with reference to FIG. 8. Specifically, an angle θ1 of an inclination surface of the first energy application portion 118 of the right convex part 112 of the first holding member 110 with respect to the horizontal surface is greater than an angle θ2 of an inclination surface of the second energy application portion 128 of the convex part 122 of the second holding member 120 with respect to the horizontal surface. By setting the angles of the inclinations as described above, materials between two joint surfaces of the held living tissues can be pressed out more efficiently.

In the first holding member 110 and the second holding member 120 of this embodiment, shearing stress occurs on the inclination surfaces of the right convex part 112 and the left convex part 114 of the first holding member 110 that face the convex part 122 of the second holding member 120. The first energy application portion 118 and the second energy application portion 128 are provided on those inclination surfaces that produce the shearing stress. Thus, since the energy application portions are provided in parts which press out contents between the first tissue 910 and the second tissue 920, high joint strength can be obtained.

In this embodiment, no energy application portion is provided on a top portion of the convex part 122 of the second holding member 120 or a bottom of the deepest portion of the first holding member 110. Contents between the first tissue 910 and the second tissue 920 in this part cannot be pressed out. Therefore, even if an energy application portion is provided in this part, a joint strength therein will be weak. Joining treatment using the treatment apparatus 20 of this embodiment is based on the assumption that a central portion of the tissues held by the treatment portion 100 will be cut after the treatment. Therefore, no energy application portion is provided in a central portion of the treatment portion 100.

As described above, the energy application portions are provided in an area as described below: A point on one holding member that is nearest to the other holding member is defined as a first point, and a point on the other holding member that is nearest to the one holding member is defined as a second point. An area defined between a trace of the second point and an extended line of the trace on one hand and a line passing through the first point and parallel to the trace and the extended line on the other is defined as a first area. The energy application portion is provided in an area at least included in the first area of at least one of the holding members.

Also in this embodiment, because of the shapes of the first holding member 110 and the second holding member 120, unwanted materials in the living tissues to be treated are pressed out and a high strength joint can be realized.

### Third Embodiment

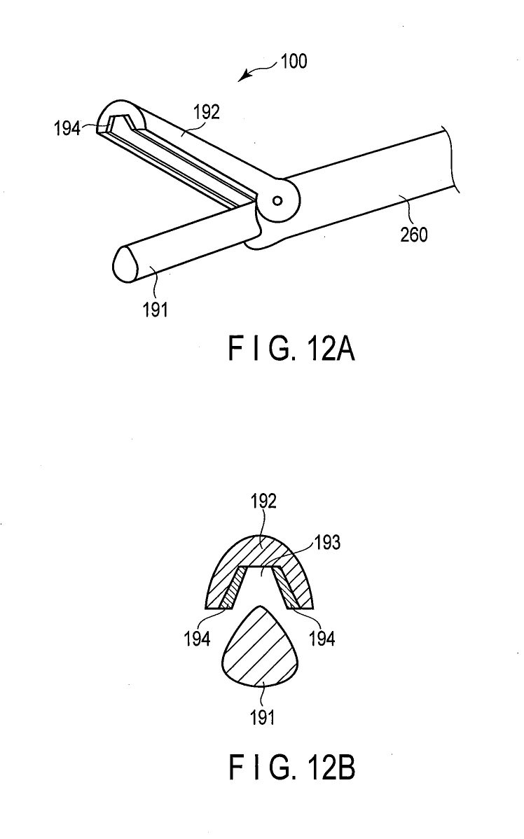

The third embodiment is explained below. In the following, matters different from the second embodiment will be explained. Identical symbols will be used for identical parts, and detailed explanations thereof will be omitted. FIG. 12A is a perspective diagram showing a treatment portion 100 of this embodiment. FIG. 12B is a cross-sectional view on a cross section perpendicular to the longitudinal direction of the treatment portion 100, as viewed from a distal end side of the treatment portion 100. In this embodiment, a ninth holding member 191 comprises an ultrasonic probe. The ninth holding member 191 has an elongated shape, and is connected through a shaft 260 to an ultrasonic vibration source provided in an operating portion main body 272. The ninth holding member 191 longitudinally vibrates in the longitudinal direction thereof. A shape of the ninth holding member 191 on a cross section perpendicular to the longitudinal direction has an approximately circular shape, as shown in FIG. 12B. The shape can easily transmit ultrasonic vibrations.

The treatment portion 100 comprises a tenth holding member 192 facing the ninth holding member 191. The tenth holding member 192 comprises a concave part 193. The ninth holding member 191 and the tenth holding member 192 have shapes so that the ninth holding member 191 fits inside the concave part 193 of the tenth holding member 192. Energy application portions 194 are provided in parts of the concave part 193 that face the ninth holding member 191. In this embodiment, the energy application portions 194 are, for example, heaters.

The treatment portion 100 holds living tissues to be treated with the ninth holding member 191 and the tenth holding member 192, and joins the living tissues by heating the living tissues with the energy application portions 194 which are the heaters. Further energy is applied to the held living tissues by ultrasonic vibrations of the ninth holding member 191 to increase the joint strength. Then, the held living tissues are cut by ultrasonic vibrations of the ninth holding member 191.

The energy application portions 194 are not limited to the heaters, but may be, for example, electrodes to apply a high-frequency voltage to the living tissues to be treated.

Also in this embodiment, because of the shapes of the ninth holding member 191 and the tenth holding member 192, unwanted materials in the living tissues to be treated are pressed out and a high strength joint can be realized.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical treatment apparatus comprising:
a first holding member; and
a second holding member configured to move relative to the first holding member and hold a living tissue between the first holding member and the second holding member,
wherein, on a cross section including a point where the first holding member and the second holding member are in contact with the living tissue, where a point on the first holding member that is nearest to the second holding member is defined as a first point and a point on the second holding member that is nearest to the first holding member is defined as a second point, the first holding member and the second holding member have shapes in which an extended line of a trace of the second point moving relative to the first holding member does not pass through the first point.

2. The medical treatment apparatus according to claim 1, wherein, of the shapes of the first holding member and the second holding member on the cross section, one has a convex part and another has a concave part, and the convex part configured to fit inside the concave part.

3. The medical treatment apparatus according to claim 2, further comprising an electrode configured to apply a high-frequency voltage to the living tissue,
wherein, when an area between the trace of the second point and the extended line of the trace, and a line passing through the first point and parallel to the trace and the extended line is defined as a first area, the electrode is provided in an area at least included in the first area of at least one of the first holding member and the second holding member.

4. The medical treatment apparatus according to claim 2, further comprising a heater configured to apply thermal energy to the living tissue,
wherein, when an area between the trace of the second point and the extended line of the trace, and a line passing through the first point and parallel to the trace and the extended line is defined as a first area, the heater is provided in an area at least included in the first area of at least one of the first holding member and the second holding member.

5. The medical treatment apparatus according to claim 2, wherein:
where an area between the trace of the second point and the extended line of the trace, and a line passing through the first point and parallel to the trace and the extended line is defined as a first area and a line perpendicular to the trace and the extended line of the trace is defined as a reference line, the shape of the first holding member is a shape having a first inclination in the first area and the shape of the second holding member is a shape having a second inclination in the first area, and
where the first inclination and the reference line forms a first angle and the second inclination and the reference line forms a second angle, the first angle and the second angle are different.

6. The medical treatment apparatus according to claim 2, wherein:
where an area between the trace of the second point and the extended line of the trace, and a line passing through the first point and parallel to the trace and the extended line is defined as a first area and a line perpendicular to the trace and the extended line of the trace is defined as a reference line, the shape of one of the first holding member and the second holding member that includes the convex part is a shape having a first inclination in the first area and the shape of the other of the first holding member and the second holding member that includes the concave part is a shape having a second inclination in the first area, and
where the first inclination and the reference line forms a first angle and the second inclination and the reference line forms a second angle, the first angle is smaller than the second angle.

7. The medical treatment apparatus according to claim 6, wherein the convex part and the concave part have a height and a depth, which are determined in accordance with a thickness of the living tissue.

8. The medical treatment apparatus according to claim 7, wherein the height and the depth are greater than twice thickness of mucous membrane included in the living tissue.

9. The medical treatment apparatus according to claim 1, further comprising an electrode configured to apply a high-frequency voltage to the living tissue,
wherein, when an area between the trace of the second point and the extended line of the trace, and a line passing through the first point and parallel to the trace and the extended line is defined as a first area, the electrode is provided in an area at least included in the first area of at least one of the first holding member and the second holding member.

10. The medical treatment apparatus according to claim 1, further comprising a heater configured to apply thermal energy to the living tissue,
wherein, when an area between the trace of the second point and the extended line of the trace, and a line passing through the first point and parallel to the trace and the extended line is defined as a first area, the heater is provided in an area at least included in the first area of at least one of the first holding member and the second holding member.

11. The medical treatment apparatus according to claim 1, wherein:
where an area between the trace of the second point and the extended line of the trace, and a line passing through the first point and parallel to the trace and the extended line is defined as a first area and a line perpendicular to the trace and the extended line of the trace is defined as a reference line, the shape of the first holding member is a shape having a first inclination in the first area and the shape of the second holding member is a shape having a second inclination in the first area, and
where the first inclination and the reference line forms a first angle and the second inclination and the reference line forms a second angle, the first angle and the second angle are different.
